(19) United States
(12) Patent Application Publication
(10) Pub. No.: US 2011/0224477 A1
(43) Pub. Date: Sep. 15, 2011

(54) THERAPEUTIC DEVICE COMBINING RADIATION THERAPY AND THERMOTHERAPY

(75) Inventor: Rolf Issels, Munchen (DE)

(73) Assignee: Helmholtz Zentrum München Deutsches Forschungszentrum für Gesundheit und Umwelt (GmbH), Neuherberg (DE)

(21) Appl. No.: 13/121,567

(22) PCT Filed: Oct. 8, 2008
(86) PCT No.: PCT/EP2008/008488

§ 371 Date: Apr. 13, 2011

(51) Int. Cl. A61N 5/02

(52) U.S. Cl. 600/2

(57) ABSTRACT

The disclosure relates to a therapeutic device for treatment of a patient, particularly for cancer treatment, which includes a radiation therapy apparatus for applying an ionizing radiation to the patient, and an integrated thermotherapeutic heating device for inducing a regional hyperthermia in the patient.

### RELATED APPLICATION

This is a §371 of International Application No. PCT/EP2008/008488, with an international filing date of Oct. 8, 2008 (WO 2010/040364 A1, published Apr. 15, 2010).

### TECHNICAL FIELD

The disclosure relates to a therapeutic device for treatment of a patient, particularly for cancer treatment.

### BACKGROUND

One of the conventional therapies against cancer is radiation therapy which uses ionizing radiation to kill cancer cells and shrink tumors. The radiation can be administered externally by external beam radiotherapy (EBRT) or internally by the so-called brachytherapy.

A further development of the external beam radiotherapy is the so-called tomotherapy where the radiation is delivered to the patient helically by a radiation source which is rotating around the patient. An advantage of the tomotherapy is the ability to precisely deliver radiation to a cancerous tumor while sparing the normal healthy tissue around it.

Another treatment against cancer is the so-called thermotherapy where a localized or regional hyperthermia is induced in the patient in the area of the cancerous tumor.

One positive effect of thermotherapy is the improvement of the blood supply and therefore the oxygenation in the cancerous tumor so that the tumor cells are more responsive to a following radiation therapy or chemotherapy.

Further, the thermotherapy impairs the ability of the cancerous tumor cells to repair damages caused by a preceding radiation therapy.

It is further known to combine radiation therapy and thermotherapy to improve the therapy results. However, it is disputed whether the thermotherapy should be administered to the cancer patient, before or after the radiation therapy.

In some clinics, the cancer patients are first subjected to a thermotherapy. Then, the cancer patients are brought to another room where the radiation therapy is administered to the patients in a specialized radiation therapy apparatus, e.g. a tomotherapy apparatus.

In other clinics, the cancer patients are first subjected to the radiation therapy, e.g. in a tomotherapy apparatus. Then, the cancer patients are brought to another room where the thermotherapy is administered to the cancer patients in order, to impair the ability of the cancerous tumor cells to repair the damages caused by the preceding radiation therapy.

However, the aforementioned conventional cancer therapies are not entirely satisfactory.

### SUMMARY

Therefore, it is desirable to provide an improved therapeutic device for treatment of cancer patients.

Further, it is desirable to provide a method of operation of such a therapeutic device.

This disclosure teaches a novel therapeutic device and a corresponding method of operation as defined in the independent claims.

The disclosure comprises the general technical teaching that the radiation therapy and the thermotherapy should be applied to the cancerous cells spatially and temporally simultaneously. Therefore, the disclosure is distinguishable from the aforementioned conventional combination therapies where the thermotherapy is administered to the cancer patients either before the radiation therapy or after the radiation therapy.

Therefore, the therapeutic device according to the disclosure comprises both a radiation therapy apparatus and an integrated thermotherapeutic heating device. The radiation therapy apparatus applies an ionizing radiation to the patient, while the thermotherapeutic heating device induces a regional hyperthermia in the patient.

The term hyperthermia defines a procedure in which the body cells in the area of treatment are heated to a temperature of 40° C.-46° C. It should further be noted that the thermotherapeutic heating device induces a regional hyperthermia only. Therefore, the hyperthermia is spatially limited to the area of treatment while the body cells outside the area of treatment remain at a lower temperature near the normal body temperature.

Further, the radiation therapy apparatus and the thermotherapeutic heating device are preferably adapted to operate simultaneously, so that a regional area of treatment within the patient can be subjected to a radiation therapy and a regional hyperthermia simultaneously.

Further, the radiation therapy apparatus preferably administers an external beam radiotherapy (EBRT) to the patient. However, the disclosure is not restricted to an external beam radiotherapy. Alternatively, the ionizing radiation can be delivered to the patient in other ways, e.g. by a probe which is inserted into the patient.

Further, the radiotherapy apparatus may administer an intensity modulated radiation therapy (IMRT) to the patient. Therefore, the radio therapy apparatus generates a dose distribution which is well adapted to the target volume (e.g. a tumor) while sparing healthy tissues according to radiobiological considerations.

It should further be noted that the radiation therapy apparatus may administer an image-guided radiation therapy (IGRT) to the patient by the use of images of the body interior of the patient, wherein the images are generated by a body scanner, e.g. a computer tomograph (CT). Preferably, X-ray beam are utilized to generate a so-called MV-CT (megavolt computer tomograph) or cone-beam CT before starting the radiotherapy. This MV-CT is preferably matched with the previously generated planning CT to correct the position of the patient for the actual radiotherapy, which is called interfractional image guidance.

Further, the radiation therapy apparatus may be a tomotherapy apparatus which is adapted for an image-guided and/or intensity modulated delivery of beams of the ionizing radiation by rotating around the patient slice-by-slice thereby employing all directions.

Further, the tomotherapy apparatus may comprise a multi-leaf collimator for matching the radiation field to the shape of the tumor by modulating the dose distribution according to the shape of the tumor. Suitable tomotherapy apparatuses are commercially available from the company Tomotherapy Inc. (USA).

The thermotherapeutic heating device may induce the regional hyperthermia within the patient by depositing ultrasound, electromagnetic waves, particularly radio frequency waves or microwaves, into the patient. The aforementioned radiation may be radiated by an antenna arrangement which may be annular or ring-shaped and surrounds the patient. The antenna arrangement may use the interference principle and surrounds the patient. Suitable thermotherapeutic heating devices are commercially available from the company BSD Medical Corporation (USA).

Alternatively, the cancerous cells can be heated by a probe which can be inserted into the patient to an area of treatment, wherein the probe locally or regionally heats the patient, in the area of treatment so that a regional hyperthermia is induced in the cells within the area of treatment.

The device for image-guidance of radiotherapy, e.g. megavolt computer tomograph (MV-CT) is also useful to control the heat treatment distribution. Basically, the MV-CT generates a three-dimensional density distribution of the patient characterized by the so-called Hounsfield units (HU) which has a direct relationship to the absorption coefficient μ of the tissue: HU(x, y)=1000(μ(x, y)/μ₀−1), wherein the reference tissue μₒ is water. The density HU is dependent on temperature, i.e. 0.45HU/° C. for muscle tissue. Therefore, a series of MV-CTs, e.g. before the heat treatment, at the end of the heat-up phase (e.g. after 20 minutes) and later during the plateau provides valuable information about the temperature distribution in addition.

Further, the therapeutic device may include at least one control unit controlling the operation of the radiation therapy apparatus and the operation of the thermotherapeutic heating device according to a predetermined program which is executed in the control unit. The predetermined program corresponds to a treatment plan which is preferably generated in a planning system.

For example, the predetermined program can define the location and the shape of the area of treatment of the thermotherapeutic heating device and/or the radiation therapy apparatus. This allows a matching of the area of treatment to the location and shape of a cancerous tumor which is to be treated.

Firstly, in the planning CT dataset the target volume (tumor and risk areas) and organs of risk are specified. The optimal dose distribution using the multi-leaf collimator (MLC) for intensity-modulation is calculated in the radiotherapy planning system.

Secondly, in a hyperthermia planning system the power deposition pattern and then the temperature distribution is calculated for any given phase and amplitude set of the antennas. Then, the particular phases and amplitudes are predicted providing the best solution fora certain tumor topography.

As a consequence, the predetermined program can define a sequence of operation of the radiation therapy apparatus and/or the thermotherapeutic heating device. In an embodiment of the disclosure, the predetermined program defines three successive phases of treatment characterized by different operational characteristics of the radiation therapy apparatus and the thermotherapeutic heating device.

In the first phase of the treatment, a MV-CT of the patient may be used to position the patient and an applicator correctly in the geometry. Then, the thermotherapeutic heating device is switched on and the radiation therapy apparatus is switched off. Therefore, the thermotherapy administered to the cancerous cells in the first phase of the treatment with suitable antenna parameters results in an improvement of the blood supply and the oxygenation of the cancerous cells so that the cancerous cells better respond to the following radiation therapy.

In the second phase of the treatment, both the thermotherapeutic heating device and the radiation therapy apparatus may be switched on, so that the cancerous cells are subjected to the ionizing radiation and the hyperthermia simultaneously. It has been found that such a simultaneous administration of the radiation therapy and the thermotherapy results in a particularly high synergism of the radiation and temperature effect in the cells.

In the third phase of treatment, the thermotherapeutic heating device is switched on and the radiation therapy apparatus is switched off. The thermotherapy administered to the patient during the third phase of the treatment avantageously impairs the ability of the cancerous cells to repair damages caused by the preceding radiation therapy.

The aforementioned three phases of treatment preferably each have a duration in a range between 5 minutes and 30 minutes and may be in a range between 15 minutes and 25 minutes. Further, the total accumulated duration of the aforementioned three phases may be in a range between 30 minutes and 90 minutes.

Further, the above-mentioned predetermined program can define the dose distribution and/or intensity of the radiation therapy and/or the control of the thermotherapy, wherein the intensity can vary according to a specific temporal profile during the treatment time.

It should further be noted that the control unit(s) may control the position of the regional areas of treatment of the radiotherapy apparatus and the thermotherapeutic heating device in such a way that the regional area of treatment of the radiotherapy apparatus spatially overlaps with the regional area of treatment of the thermotherapeutic heating device. Therefore, the cells in the overlapping area (e.g. the tumor infiltrated tissue) are subjected both to a radiation therapy and a thermotherapy simultaneously.

Further, the therapeutic device may comprise an integrated body scanner generating images of the body interior of the patient, which is important for the aforementioned image-guided radiation therapy (IGRT). The body scanner may be a computer tomograph (CT) although other types of conventional body scanners can be used, e.g. a magnet, resonance tomograph (MRT), a positron emission tomograph (PET), an X-ray apparatus, an ultrasonograph or an ultrasound tomograph. Further, the body scanner can be a combination of the aforementioned imaging devices wherein the images of the different imaging devices are fused. For example, an image fusion is possible of the images generated by a computer tomograph (CT) on the one hand and the corresponding images of a magnet resonance tomograph (MRT) on the other hand.

Further, the integrated MV-CT can be performed with a slice thickness of 2-15 mm. The data acquisition for one slice (half rotation) needs 10 s. Therefore, a volume of 10-30 cm length can be scanned in 1-3 minutes for a large slice thickness of 15 mm (and for better spatial resolution with larger acquisition times accordingly). While the initial MV-CT is employed for image-guidance of radiotherapy (IGRT, interfractional on-line correction) a temporal sequence of MV-CT can be used to monitor and control the temperature distribution. Note that the tomotherapy must be interrupted for the MV-CT, bin a well-defined pause of irradiation for some minutes is possible. In any CT (kVCT or MV-CT) there is a competition between spatial and temperature resolution. Clearly, the standard deviation of HU (and therefore the contrast resolution) is much better for kV-CT (±5 HU for a 2 mm pixel size at 140 kV) than for MV-CT (±15 HU for a 1-2 cm pixel size at 3 MV).

For any object of the size of one pixel, the low contrast resolution is by far not sufficient to provide a reasonable temperature resolution (derived from 0.45 HU/° C.). However, spatial integration of pixels over several slices and in the slice, i.e. in all three coordinates, can reduce the standard deviation of the contrast resolution considerably. We find the following potentials from statistical considerations: a) For voxel sizes of 3-5 cm, we achieve a temperature resolution of ±6° C., i.e. we are able to detect areas of 3-5 cm extension with >50° C. (hot spot detection). b) For voxel sizes of 5-10 cm, we achieve a temperature resolution of better ±3° C., we are able to verify an effective mean temperature of 43° C. in a tumor region and to trace a high (probably too high) temperature level around 43° C. in normal tissue such as muscles and fat. The temperature analysis is performed in this way by postprocessing of the MV-CT dataset which, is transferred in a standard DICOM (Digital Images and Communications in Medicine) format into the hyperthermia planning program.

Moreover, the disclosure includes protection for a corresponding method of operation of the aforementioned therapeutic device for cancer treatment.

Finally, the disclosure also includes protection for therapeutic radiation which is a combination of two different types of radiation. The first type of radiation is an ionizing radiation which is suitable for the conventional radiation therapy of a patient. The Second type of radiation is a radio frequency radiation which is suitable for thermotherapeutically inducing a regional hyperthermia within the patient.

The disclosure and its particular features will become more apparent from the following detailed description considered with reference to the accompanying drawings.

### BRIEF DESCRIPTION OF THE DRAWINGS

FIG. 1 is a schematical perspective view of a therapeutic device according to the disclosure combining a tomotherapy apparatus and a thermotherapeutic heating device,

FIG. 2 is a schematic cross section of the therapeutic device of FIG. 1,

FIG. 3 is a schematic block diagram of the therapeutic device shown in FIG. 1,

FIGS. 4A and 4B show a flow chart illustrating the mode of operation of the therapeutic device shown in FIGS. 1 to 3.

### DETAILED DESCRIPTION

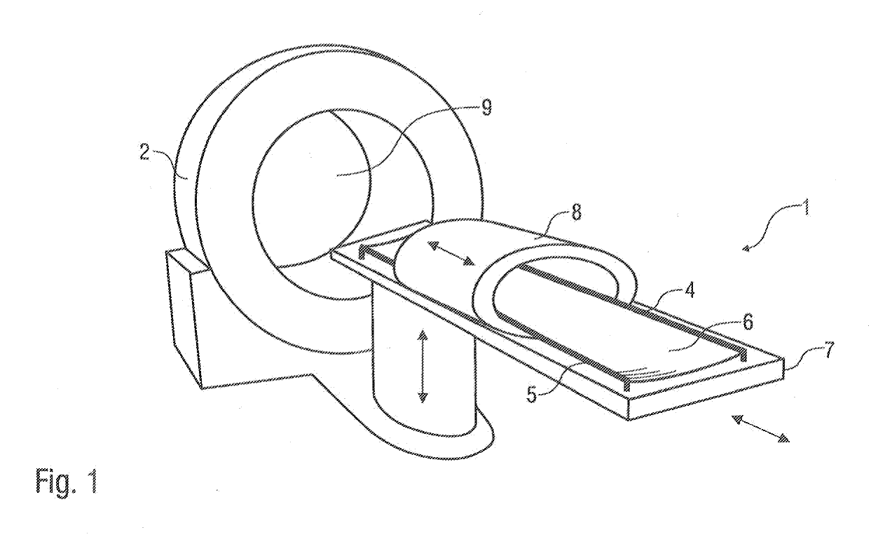

FIGS. 1-3 illustrate a novel therapeutic device 1 combining radiation therapy and thermotherapy for the treatment of cancer.

Firstly, the therapeutic device 1 comprises a tomotherapy apparatus 2 which administers a tomotherapy to a tumor. The tomotherapy apparatus 2 can be based on a conventional tomotherapy apparatus which is commercially available from the company Tomotherapy Inc. (USA).

The tomotherapy apparatus 2 comprises a treatment table 3 with two lateral metal rods 4, 5 on the top side of the treatment table 3, wherein a mat 6 is spanned between the rods 4, 5 so that a patient 7 can rest on the mat 6 during treatment.

Further, a thermotherapeutic heating device 8 is slidably mounted on the treatment table 3. Before the beginning of the treatment, the thermotherapeutic heating device 8 is moved to the foot end of the treatment table 3 facing the tomotherapy apparatus 2, so that the patient 7 can easily lie down on the mat 6 of the treatment table 3. Then, the thermotherapeutic heating device 8 is moved back to the treatment position in which it surrounds the patient 7 lying on the mat 6.

After the aforementioned positioning of the patient 7, the treatment table 3 is moved into an aperture 9 of the tomotherapy apparatus 2 so that the thermotherapeutic heating device 8 is coaxially aligned and centrally located in the aperture 9 of the tomotherapy apparatus 2. In this embodiment, the aperture 9 has a diameter of d=85 cm so that the entire treatment table 3 with the patient 7 and the thermotherapeutic heating device 8 can be moved into the aperture 9 of the tomotherapy apparatus 2.

The integrated thermotherapeutic heating device 8 administers a thermotherapy to the cancer patient 7, so that a regional hyperthermia is induced in the patient 7 in the area of treatment. The thermotherapeutic heating device 8 can be based on a conventional thermotherapeutic heating device which is commercially available from the company BSD Medical Corporation (USA).

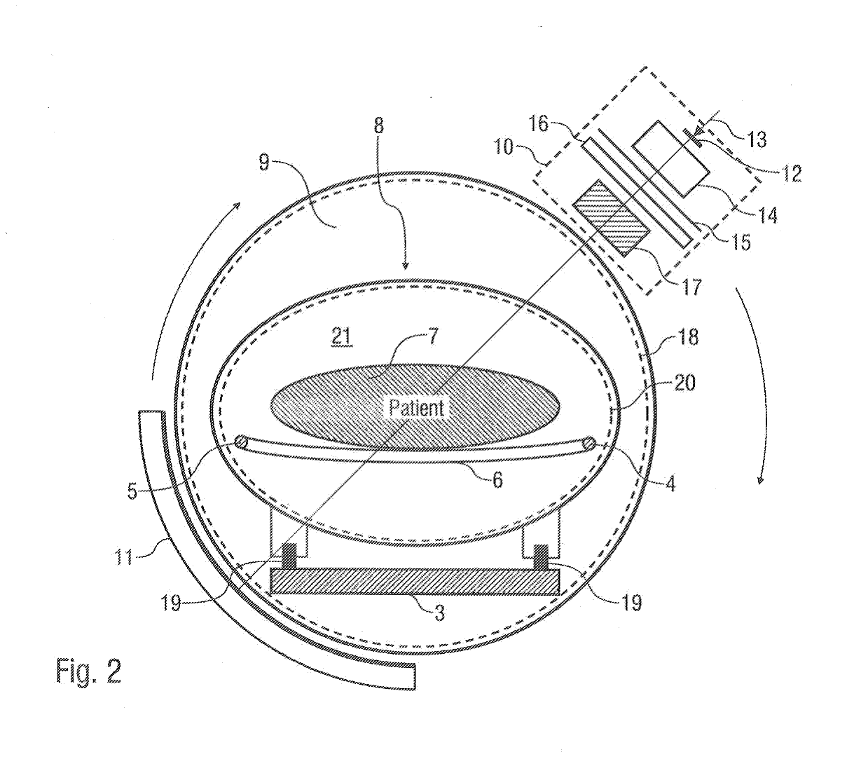

Reference is now made to the cross section shown in FIG. 2 showing further details of the thermotherapeutic heating device 8 and the tomotherapy apparatus 2.

The tomotherapy apparatus 2 comprises a radiation unit 10 and a radiation detector 11 which are arranged opposite to each other and rotating around the patient 7. The radiation unit 10 comprises a high-density metallic target 12 (e.g. tungsten) which is hit by an electron beam 13 of 6 MeV generated by a linear accelerator (LINAC: Linear accelerator) so that a 6 MeV X-ray beam is generated. Further, the radiation unit 10 comprises a primary collimator 14, a flattening filter 15, an ion chamber 16 and a multi-leaf collimator 17 (MLC: Multi-leaf collimator). The primary collimator 14 collimates the X-ray beam in longitudinal direction and the flattening filter 15 homogenizes the X-ray beam. Further, the multi-leaf collimator 17 modulates the dose distribution in the x-z-plane.

Moreover, the inner circumferential wall of the aperture 9 of the tomotherapy apparatus 2 is covered by an electric shielding 18, e.g. a thin copper foil or mesh, which is shielding the electronic components of the tomotherapy apparatus 2.

Moreover, it can be seen that the thermotherapeutic heating device 8 is supported on rolls 19, so that the thermotherapeutic heating device 8 can be rolled along the treatment table 3 to facilitate the positioning of the patient 7 on the mat 6.

Further, it can be seen that the thermotherapeutic heating device 8 comprises an antenna arrangement 20 which is fed by high-frequency cables (not shown). Further, water tubes (not shown) discharge into the interior of the thermotherapeutic heating device 8 to fill the interior with a water bolus 21.

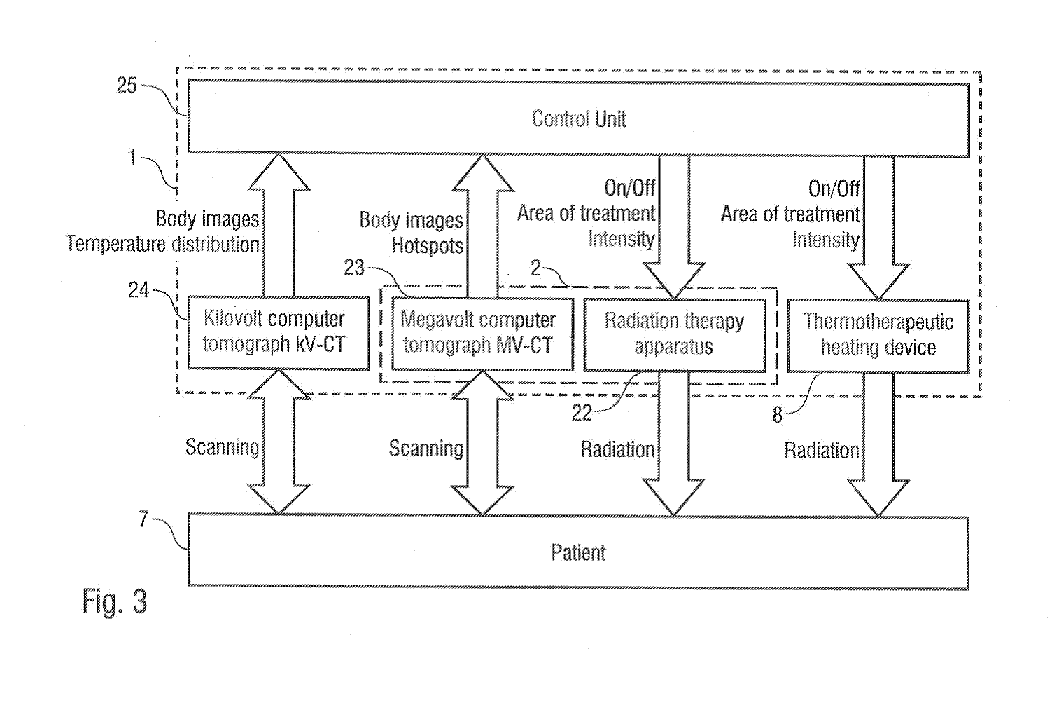

Reference is now made to FIG. 3 showing a schematic block diagram of the therapeutic device 1 for cancer treatment of the patient 7.

The block diagram shows that the tomotherapy apparatus 2 essentially consists of a radiation therapy apparatus 22 and a megavolt computer tomograph 23 (MV-CT) which generates sectional images of the patient 7 slice-by-slice.

The megavolt computer tomograph 23 is suitable to detect thermal hot spots in the patient 7 which should be avoided during the therapy.

Further, the therapeutic device 1 comprises a kilovolt computer tomograph 24 (kV-CT) which also generates sectional images of the patient 7 slice-by-slice. However, the kilovolt computer tomograph 24 has a much higher spatial resolution than the megavolt computer tomograph 23 so that the temperature distribution in the patient 7 can be determined with high precision.

Further, the therapeutic device i comprises a control unit 25 which controls the operation of the radiation therapy apparatus 22 and the thermotherapeutic heating device 8. For example, the control unit 25 either activates or inactivates the radiation therapy apparatus 22 and the thermotherapeutic heating device 8, respectively. Further, the control unit 25 defines the position and shape of the area of treatment of the radiation therapy apparatus 22 and the thermotherapeutic heating device 8 so that the areas of treatment can be matched to the position and shape of the tumor. Moreover, the control unit 25 can modulate the intensity of the radiation which is applied by the radiation therapy apparatus 22 and the thermotherapeutic heating device 8.

The control unit 25 performs an image-guided control of the radiation therapy apparatus 22 and the thermotherapeutic heating device 8, wherein the images of the patient 7 are provided by the kilovolt computer tomograph 24 and the megavolt computer tomograph 23.

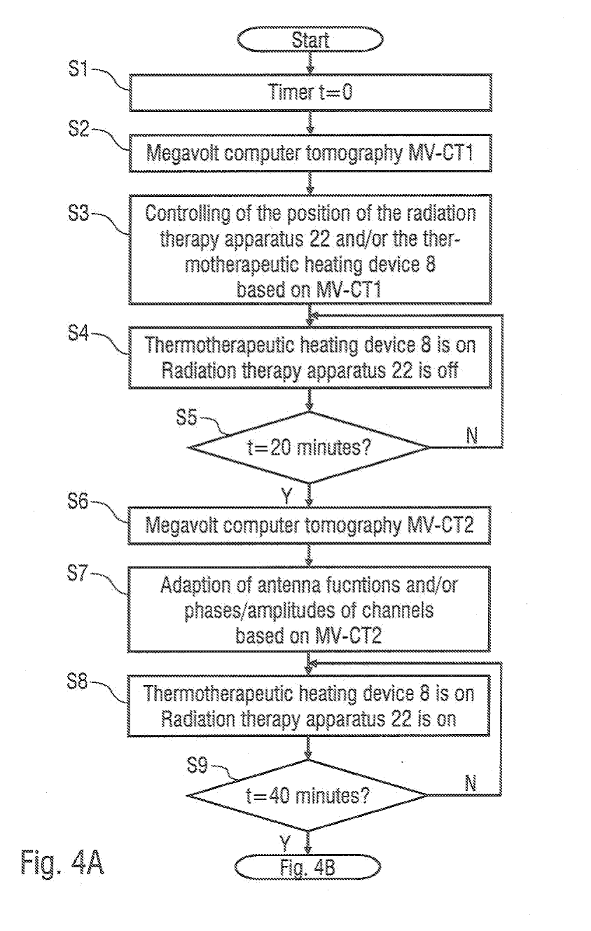

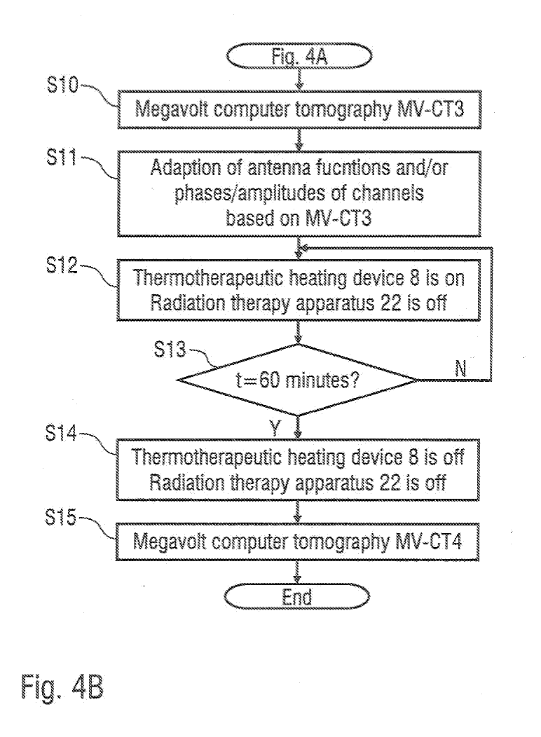

In the following, reference is made to the flowchart shown in FIGS. 4A and 4B.

Before the beginning of a therapy, the patient 7 is placed on the aforementioned treatment table 3, so that the thermotherapeutic heating device 8 surrounds the body of the patient 7.

Then, the treatment table 3 along with the patient 7 and the thermotherapeutic heating device 8 is moved into the aperture 9 of the tomotherapy apparatus 2 until the thermotherapeutic heating device 8 is positioned within the aperture 9 of the tomotherapy apparatus 2.

After these preparatory operations, the following treatment is started, wherein the operation of the tomotherapy apparatus 2 and the thermotherapeutic heating device 8 is controlled by a predetermined program which is executed in the control unit 25.

In a first step S1 before the beginning of the treatment, a timer t is resetted.

Then, a first megavolt computer tomography MV-CT1 is performed in a step S2.

The images generated by the first megavolt computer tomography MV-CT1 are then utilized in a step S3 to control and adjust the position of the patient 7 and the thermotherapeutic heating device 8 relative to each other and with regard to the tomotherapy apparatus 2.

In a next step S4, the control unit 25 activates the thermotherapeutic heating device 8 while the radiation therapy apparatus 22 keeps inactive.

In another step S5, the control unit 25 continuously checks whether a first phase of treatment of t=20 minutes already expired.

If the first phase of t=20 minutes has not yet expired, the control unit 25 continues with the step S4 in which the thermotherapeutic heating device 8 is switched on while the radiation therapy apparatus 22 is switched off.

Otherwise, the operation continues with a step S6 in which a second megavolt computer tomography MV-CT2 is conducted. The images generated by the second megavolt computer tomography MV-CT2 are then used in a step S7 to adapt the antenna functions and phases/amplitudes of channels of the therapeutic device 1.

Then, the radiation therapy apparatus 22 is switched on in step S8, while the thermotherapeutic heating device 8 keeps activated. Therefore, the therapeutic heating device 1 administers both a thermotherapy and a radiation therapy to the patient 7.

In a next step S9, the control unit 25 continuously checks whether the second phase of treatment already expired after t=40 minutes.

If so, the control unit 25 continues with step S10 in which a third megavolt computer tomography MV-CT3 is conducted.

The results of the third megavolt computer tomography MV-CT3 are then used in step S11 to adapt the antenna functions and the phases/amplitudes of the channels.

Then, in step S12 the radiation therapy apparatus 22 is switched off while the thermotherapeutic heating device 8 keeps activated.

Further, the control unit 25 continuously checks in step S13 whether the third phase of the treatment already expired after t=60 minutes.

If so, the control unit 25 continues with a step S14 in which both the radiation therapy apparatus 22 and the thermotherapeutic heating device 8 are switched off.

In a final step S15 a fourth megavolt computer tomography MV-CT4 is conducted.

In the following, the operation of the therapeutic device 1 is described in other words to further clarify the disclosure.

The multimodal cancer treatment performs at first an image-guided and intensity modulated radiotherapy with optimal concentration of the dose in a target (here a locally advanced tumor disease) and well defined sparing of normal tissues and particular critical organs. Hereby, the treatment head, i.e. the radiation unit 10, of the LINAC rotates around the patient 7 (in the RF applicator) requiring approximately one minute per rotation and modulating the dose by switching the leafs on and of every 5°. The multi-leaf collimator 17 consists of 64 leaves with each leaf width 6.25 mm in the isocenter covering a field of 40 cm. The dose distribution is generated in one slice per rotation adjusting slice thickness between 0.5 to 5 cm. Therefore, a volume of 30 cm in longitudinal direction can be irradiated in 6 minutes (5 cm slices) to 60 minutes (0.5 cm slices).

The exact position of the patient 7 relative to the LINAC is controlled using the megavolt computer tomography (MV-CT). The imaging is performed using a detector array of 738 xenon detectors opposite to the central beam of the LINAC. The treatment CT needs 12 s per slice, i.e. 1 to 5 minutes for the whole volume. Then, the planning kilovolt computer tomography (kV-CT) dataset is fused with the treatment MV-CT dataset, and a shift vector to correct the position is determined. Note that the planning CT dataset for the patient 7 must be performed in the treatment position, i.e. in the RF applicator. Modern CT scanners for the radiotherapy planning have also gantries of 85 cm and enough place to scan a patient positioned in an RF applicator.

After repositioning, the regional hyperthermia is performed in the elliptical RF applicator (modality 1) and the IGRT and IMRT (modality 2) is performed simultaneously with the heat delivery. Typically, a heat-up phase of 20 minutes is required to obtain a plateau for the temperature distribution. Then, a second MV-CT can be performed and further MV-CT during heat delivery in order to characterized the temperature distribution. Therefore, the MV-CT is not only useful for interfractional positioning control with respect to radiotherapy, but also for online control and optimization of the temperature distribution.

Finally, it is important to note that the radio therapy is administered in a conventional fractionation, e.g. 30×2Gy. As a consequence, the thermotherapy can be applied up to 30 times simultaneously. Of course, other combinations of radio therapy and, thermotherapy are to consider. Further, a combination with radio chemotherapy is possible.

In the following, the specific characteristics of kV-CT and MV-CT are explained.

The contrast resolution is ≈5 HU (Hounsfield units) for 2 mm voxel in kV-CT. The HU are directly related to the absorption coefficient HU=1000(μ/μ₀−1). The temperature dependency of density transforms to 0.45 HU/° C. in muscle (or tumor). The standard fluctuation in one voxel is related to the standard fluctuation σ_{N} in N voxels by σ_{N}=σ(1+N)^{-3/4}.

As a consequence, we expect in a conventional CT scanner (140 kV) a temperature resolution of only 15° C. in a typical voxel of 2 mm size. However, in a voxel of 1 cm size, the resolution is improved by a factor of 126^{-3/4}, i.e. 37. Therefore, using conventional CT, the temperature resolution is better 0.5° C. in 1 cm voxels (and can be better 0.1° C. in 2 cm voxels).

The contrast resolution in MV-CT (3 MV X-rays) is described as 15 HU (1.5%) in 3 cm sized voxels for a non-neglectable dose exposition of 10-12cGy (100-120 mSv). This is a temperature resolution of only 30° C. For 6 cm objects, we obtain a temperature resolution of 6.5° C., we can differentiate, between regions 37-44° C. and regions >44° C. This is sufficient to detect hot spots >44° C. of some extension (>5 cm).

For larger objects of 9 cm extension we can even improve the temperature resolution of the mean temperature down to <3° C., i.e. we can differentiate temperature ranges 37-40° C., 40-43° C. and above 43° C. (for larger regions ≈9 cm). This is already sufficient to utilize a code which has been developed to adapt antenna functions of the hyperthermia applicator and to improve (optimize) the temperature distribution according to any prescribed objective function. This algorithm has been originally developed for MR-controlled regional hyperthermia (see Weihrauch et al. 2007 med. Phys.) but can be employed accordingly for a CT-based control.

Then, there are the following options to implement a CT-control for multi-antenna RF-hyperthermia:

Firstly, thermal hot spots can be detected, and the temperature distribution can be optimized by using. MV-CT at 3 MV (e.g. 3° C. for 9 cm objects).

Another option is an online (real-time) optimization of the heat treatment using the planning kV-CT (140 kV) with much better resolution (<0.5° C. in 1 cm objects).

Finally, a kV-CT can be integrated into the tomotherapy apparatus. Then, the onboard CT can be used for a real-time optimization as mentioned above.

### LIST OF REFERENCE MATERIALS

1 Therapeutic device
2 Tomotherapy apparatus
3 Treatment table
4 Rod
5 Rod
6 Mat
7 Patient
8 Thermotherapeutic heating device
9 Aperture
10 Radiation unit
11 Radiation detector
12 Target
13 Electron beam
14 Primary collimator
15 Flattening filter
16 Ion chamber
17 Multi-leaf collimator
18 Shielding
19 Rolls
20 Antenna arrangement
21 Water bolus
22 Radiation therapy apparatus
23 Megavolt computer tomograph
24 Kilovolt computer tomograph
25 Control unit

The foregoing examples are provided merely for the purpose of explanation and are in no way to be construed as limiting, while reference to various embodiments are shown, the words used herein are words of description and illustration, rather than words of limitation. Further, although reference to particular means, materials, and embodiments are shown, there is no limitation to the particulars disclosed herein. Rather, the embodiments extend to all functionality equivalent structure, methods, and uses, such as are within the scope of the appended claims.

1-23. (canceled)

24. Therapeutic device for treatment of a patient, comprising:
a) a radiation therapy apparatus for applying an ionizing radiation to the patient, and
b) an integrated thermotherapeutic heating device for including a regional hyperthermia in the patient.

25. Therapeutic device according to claim 24, wherein the radiation therapy apparatus and the thermotherapeutic heating device are adapted to operate simultaneously, so that a regional area of treatment within the patient can be subjected to a radiation therapy and a regional hyperthermia simultaneously.

26. Therapeutic device according to claim 24, wherein the radiation therapy apparatus administers an external beam radiotherapy to the patient.

27. Therapeutic device according to claim 24, wherein the radiation therapy apparatus administers an intensity modulated radiation therapy to the patient.

28. Therapeutic device according to claim 24, wherein the radiation therapy apparatus administers an image guided radiation therapy to the patient.

29. Therapeutic device according to claim 24, wherein the radiation therapy apparatus comprises a multi-leaf collimator for matching the radiation field to the shape of a tumor.

30. Therapeutic device according to claim 24, wherein the radiation therapy apparatus is a tomotherapy apparatus for an image guided delivery of beams of the ionizing radiation helically to the patient from different directions.

31. Therapeutic device according to claim 24, wherein the thermotherapeutic heating device:
a) induces the regional hyperthermia by radiating electromagnetic waves into the patient, and
b) comprises an antenna arrangement for radiating the electromagnetic waves into the patient.

32. Therapeutic device according to claim 31, wherein the antenna arrangement is annular or ring-shaped and surrounds the patient.

33. Therapeutic device according to claim 24, wherein the thermotherapeutic heating device comprises a probe which can be inserted into the patient to an area of treatment, wherein the probe locally heats the patient in the area of treatment.

34. Therapeutic device according to claim 24, further comprising a control unit controlling the operation of the radiation therapy apparatus and the thermotherapeutic device according to a predetermined program which is executed in the control unit.

35. Therapeutic device according to claim 34, wherein:
a) the program provides a first phase during which the thermotherapeutic heating device is, switched, on and the radiation therapy apparatus is switched off,
b) the program provides a second phase following the first phase, wherein both the thermotherapeutic heating device and the radiation therapy apparatus are switched on during the second phase, and
c) the program provides a third phase following the second phase, wherein the thermotherapeutic heating device is switched on and the radiation therapy apparatus is switched off during the third phase.

36. Therapeutic device according to claim 35, wherein:
a) the first phase, the second phase and the third phase of the treatment each have a duration in a range between 5 minutes and 30 minutes, and
b) the first phase, the second phase and the third phase have a total accumulated duration in a range between 30 minutes and 90 minutes.

37. Therapeutic device according to claim 35, wherein the control unit controls the position of the regional areas of treatment of the radiation therapy apparatus and the thermotherapeutic heating device in such a way that the regional area of treatment of the radio therapy apparatus spatially overlaps with the regional area of treatment of the thermotherapeutic heating device.

38. Therapeutic device according to claim 24, further comprising a body scanner generating images of the body interior of the patient.

39. Therapeutic device according to claim 38, wherein the body scanner is selected from a group consisting of
a) a computer tomograph,
b) a magnet resonance tomograph,
c) a positron emission tomograph,
d) an X-ray apparatus,
e) an ultrasonograph,
f) an ultrasound tomograph, and
g) a combination of different imaging devices as specified above under a) to f), wherein the images of the different imaging devices are fused.

40. Therapeutic device according to claim 24, further comprising:
a) a megavolt kilovolt computer tomograph generating body images of the patient for detecting temperature hotspots caused by the thermotherapeutic heating device, and
b) a kilovolt computer tomograph generating body images of the patient for a high-resolution determination of the temperature distribution within the patient.

41. Therapeutic device according to claim 40, wherein the control unit controls the thermotherapeutic heating device based on the images generated by the megavolt/kilovolt computer tomograph.

42. Therapeutic device according to claim 41, wherein the control unit performs an image guided control of the thermotherapeutic heating device in such a way that temperature hotspots are avoided or at least resolved.

43. Therapeutic device according to claim 41, wherein the control unit performs an image guided control of the thermotherapeutic heating device such that the actual spatial temperature distribution approximates a desired spatial temperature distribution.

44. Therapeutic device according to claim 41, wherein the control unit performs an image guided control of the radiation therapy apparatus such that the actual spatial dose distribution approximates a desired spatial dose distribution.

45. Therapeutic device according to claim 41, wherein the thermotherapeutic heating device and the radiation therapy apparatus are controlled in real-time and during the therapy.

46. Therapeutic device for treatment of a patient, comprising:
a) a radiation therapy apparatus for applying an ionizing radiation to the patient,
b) an integrated thermotherapeutic heating device for inducing a regional hyperthermia in the patient,
c) a control unit controlling the operation of the radiation therapy apparatus and the thermotherapeutic device according to a predetermined program which is executed in the control unit, and
d) a body scanner generating body images of the patient for a high-resolution determination of the temperature distribution within the patient,
e) wherein the control unit performs an image guided control of the thermotherapeutic heating device in such a way that
e1) temperature hotspots are avoided or at least resolved and/or
e2) the actual spatial temperature distribution approximates a desired spatial temperature distribution.

47. Method of operation of a therapeutic device for treatment of a patient, comprising the following steps:
a) activating a radio therapy apparatus so that the patient is subjected to a radio therapy, wherein the radio therapy apparatus is activated by a control unit, and
b) activating an integrated thermotherapeutic heating device for inducing a regional hyperthermia in the patient, wherein the thermotherapeutic heating device is activated by the control unit.

48. Method according to claim 47, wherein the control unit controls:
a) the activation and deactivation of the radio therapy apparatus and the thermotherapeutic heating device according to a predetermined program which is running on the control unit, and
b) the position of the regional areas of treatment of the radio therapy apparatus and the thermotherapeutic heating device.

49. Method according to claim 48, wherein:
a) the program provides a first phase during which the thermotherapeutic heating device is switched on and the radiation therapy apparatus is switched off,
b) the program provides a second phase following the first phase, wherein both the thermotherapeutic heating device and the radiation therapy apparatus are switched on during the second phase,
c) the program provides a third phase following the second phase, wherein the thermotherapeutic heating device is switched on and the radiation therapy apparatus is switched off during the third phase.

50. Method according to claim 49, wherein:
a) the first phase, the second phase and the third phase of the treatment each have a duration in a range between 5 minutes and 30 minutes, and
b) the first phase, the second phase and the third phase have a total accumulated duration in a range between 30 minutes and 90 minutes.

51. Method according to claim 26, wherein the control unit controls the position of the regional areas of treatment of the radio therapy apparatus and the thermotherapeutic heating device such that the regional area of treatment of the radio therapy apparatus spatially overlaps with the regional are of treatment of the thermotherapeutic heating device.

52. Method according to claim 49, further comprising the following step:
generating body images of, the patient using a megavolt/kilovolt computer tomograph.

53. Method according to claim 52, further comprising the following step:
controlling the thermotherapeutic heating device based on the images generated by the megavolt/kilovolt computer tomograph.

54. Method according to claim 53, further comprising the following step:
image guided control of the thermotherapeutic heating device such that temperature hotspots are avoided or at least resolved.

55. Method according to claim 53, further comprising the following step:
image guided control of the thermotherapeutic heating device such that the actual spatial temperature distribution approximates a desired spatial temperature distribution.

56. Method according to claim 53, further comprising the following step:
image guided control of the radiation therapy apparatus such that the actual spatial dose distribution approximates a desired spatial dose distribution.

57. Therapeutic radiation comprising an ionizing radiation which is suitable for radiation therapy of a patient, further comprising a radio frequency radiation which is suitable for thermotherapeutically inducing a regional hyperthermia within the patient.
